(19) United States
(12) Patent Application Publication
(10) Pub. No.: US 2019/0380626 A1
(43) Pub. Date: Dec. 19, 2019

(54) MULTIMODAL SYSTEMS AND METHODS FOR DETECTING AND QUANTITATING CELL OR OTHER PARTICLE TARGETS IN A BLOODSTREAM OF A LIVING BEING

(71) Applicant: IVDIAGNOSTICS, INC., Crown Point, IN (US)

(72) Inventors: Frank SZCZEPANSKI, Valparaiso, IN (US); Christopher CLEMENS, Crown Point, IN (US); Joseph FIRCA, Lake Bluff, IL (US)

(73) Assignee: Ivdiagnostics, Inc., Crown Point, IN (US)

(21) Appl. No.: 15/753,431

(22) PCT Filed: Aug. 18, 2016
(86) PCT No.: PCT/US2016/047504

§ 371 Date: Jul. 16, 2018

Related U.S. Application Data
(60) Provisional application No. 62/206,502, filed on Aug. 18, 2015.

(51) Int. Cl. A61B 5/1455; A61B 5/00; G01N 33/49

(52) U.S. Cl. CPC A61B 5/1455; A61B 5/0071; A61B 5/6838; A61B 2503/42; G01N 33/49; A61B 2503/40; A61B 5/4848

(57) ABSTRACT

Provided is a device for excitation of a fluorescent component associated with a predetermined target circulating in blood flowing through a blood vessel of a living being. The device includes a clamping device, a coherent light source that emits light causing excitation of the fluorescent component forming a conjugate with the predetermined target in the blood stream. A fluorescence sensor senses and measures a fluorescent effect exhibited by the fluorescent component forming a portion of the conjugate in response to excitation of the fluorescent component by the light emitted by the coherent light source. Optical fibers transmit the emitted light between the light source and the clamping device, and the fluorescent effect exhibited by the fluorescent component of the conjugate between the clamping device and the sensor.

### CROSS-REFERENCE TI) RELATED APPLICATIONS

This application claims the benefit of U.S. Provisional Application No. 62/206,502, filed Aug. 18, 2015, which is incorporated in its entirety herein by reference.

### BACKGROUND OF THE INVENTION

### 1. Field of the Invention

This application relates generally to a method and apparatus for the in vivo detection of particle targets in blood and, more specifically, to a minimally-invasive apparatus and method for detecting and quantitating fluorescently-labeled cells or other particles in the bloodstream of humans and animals in vivo.

### 2. Description of Related Art

Current systems available for detecting cells or other particle targets in the bloodstream, such as the Cell Search Circulating Tumor Cell Test (Janssen Diagnostics, LLC) and the MACS Whole Blood Analysis System (Miltenyi Biotec), require that a blood sample be invasively drawn from the subject prior to analysis, followed by sample processing, and final analysis of the results external to the subject by flow cytometry or similar methods. These systems have drawbacks, as they are specifically adapted for, and limited to use with human clinical patients, are invasive, require lengthy sample processing times, can only detect cancer and blood cells, and are not configured for research use with laboratory animals.

### BRIEF SUMMARY OF THE INVENTION

It would be advantageous to provide multimodal systems and methods for detecting and quantitating cell or other particle targets in the bloodstream of humans and laboratory animals. Application of this technology would provide wide-ranging benefits within the fields of clinical medicine and biomedical research. According to one aspect, the subject invention relates generally to a system including instrumentation and. minimally-invasive methods for detecting and quantitating fluorescently-labeled cells or other particles (“targets”) in the bloodstream of human patients and animals (“subjects) in vivo, possibly without the necessity of drawing blood at all. Various examples of the systems and methods are provided herein.

The system and methods described herein have utility within the fields of clinical medicine and biomedical research by providing a means for the in vivo detection and quantitation of targets including, but not limited to, circulating tumor cells (CTCs), other cell types of human or animal origin, protozoa, fungal cells, bacteria, viruses, and fluorescent microparticles, in human patients, animal hosts and laboratory models. Thusly, the present invention provides clinicians and researchers a valuable tool for detecting, quantitating and monitoring target concentrations in the bloodstream of living subjects.

According to another aspect, the present invention provides a system by which fluorescently-labeled targets can be detected and quantitated in vivo, in a non-invasive or minimally-invasive manner, in the bloodstream of living subjects. This system negates the time-consuming need to invasively obtain and process a sample of blood or other body fluid from the subject, and allows the analysis to be conducted without disruption of the circulatory system or causing harm, stress or discomfort to the patient or subject animal. One benefit of such a system is that it allows the data to be monitored and analyzed in real time, with no delay due to sample extraction and processing outside of the test subject. In addition to providing the benefit of allowing the analysis to be conducted in vivo, the present invention allows multiple analyses to be conducted on the subject over an extended time period, enabling comparison of data points collected over time to be made.

Practical applications of the present invention in human clinical patients and laboratory animals include, but are not limited to, diagnosis and monitoring of disease progression and treatment effectiveness in cancer patients, diagnosis and monitoring of disease progression and treatment effectiveness in infectious disease patients, studies of carcinogenesis and cancer metastasis, anti-cancer agent efficacy studies, study of infectious disease processes, evaluation of treatments for infectious diseases, diagnosis and study of progression and treatment of immune system disorders, pharmacokinetic studies, toxicology studies, and studies of the circulatory system.

According to another aspect, the present technology includes a system which generates a coherent light signal of a specific wavelength which is conducted, via a fiber optic cable bundle, to the exterior surface of a subcutaneous blood vessel of the subject. The distal end of the fiber optic cable bundle (“sensor tip”) is placed on the skin surface, directly over and substantially perpendicular to, the blood vessel, and is secured in place by use of an optical interface clamp device. The light signal emitted from the sensor tip penetrates the skin and passes through the vessel wall into the vessel lumen, and in so doing illuminates the subject's blood and any particles which it contains as they flow past the sensor tip. Any particles passing through the vessel which have been previously labeled with, or contain a fluorescent marker, which responds to the impinging wavelength of excitation will fluoresce by emission of a characteristic wavelength of light as they pass by the sensor tip. The resulting emitted light is then conducted from the sensor tip, via a second set of fibers in the fiber optic cable bundle, to a detector and signal amplification device which registers and records each fluorescent signal corresponding to a passing fluorescent particle. Thusly, the present invention detects and quantitates fluorescently-labeled targets in the bloodstream of the subject in vivo.

For example, a conjugate comprising a fluorescent dye and an antibody (“fluorescent conjugate”) which specifically binds to a unique cancer biomarker could be injected intravenously (IV) into a human cancer patient. The fluorescent conjugate would then bind to, and label, the circulating tumor cells (CTCs) in the patient's bloodstream. The present invention could then be used to detect, quantitate, and monitor the number of CTCs remaining in the bloodstream of the patient following treatment with an anti-cancer therapy. In so doing, the efficacy of the anti-cancer treatment can be accurately assessed, thus providing the clinician with valuable information for the direction of the most effective future course of treatment in the patient.

In another application, tumor cells expressing a unique cancer biomarker could be injected IV into a laboratory mouse to establish a tumor graft. A fluorescent conjugate comprising a fluorescent dye and an antibody which specifically binds to the unique cancer biomarker could then be injected IV into the animal. The fluorescent conjugate would bind to, and label, the circulating tumor cells (CTCs). The present technology could then be used to detect, quantitate, and monitor the number of CTCs remaining in the bloodstream of the mouse following treatment with an anti-cancer agent. In so doing, the in vivo efficacy of the anti-cancer agent could be accurately assessed.

In another application, the present technology could be used to monitor a blood-borne infection and the efficacy of an anti-infective agent in a human patient or animal model of infection. In this application, the fluorescent conjugate would contain an antibody or other ligand which would specifically bind to a targetable surface receptor on the infectious agent in the bloodstream of the subject. Applicable infectious agents include, but are not limited to, bacteria, viruses, fungal cells, and protozoa. The system could then be used to monitor the effect of treatment on the numbers of circulating infectious cells in the bloodstream.

The above summary presents a simplified summary in order to provide a basic understanding of some aspects of the systems and/or methods discussed herein. This summary is not an extensive overview of the systems and/or methods discussed herein. It is not intended to identify key/critical elements or to delineate the scope of such systems and/or methods. Its sole purpose is to present some concepts in a simplified form as a prelude to the more detailed description that is presented later.

### BRIEF DESCRIPTION OF SEVERAL VIEWS OF THE DRAWING

The invention may take physical form in certain parts and arrangement of parts, embodiments of which will be described in detail in this specification and illustrated in the accompanying drawings which form a part hereof and wherein:

FIG. 1 shows a schematic representation of an illumination device in accordance with an embodiment of the present technology;

FIG. 2 shows a sectional view of a bifurcated fiber optic cable bundle taken along line 2-2 in FIG. 1;

FIG. 3 shows an illustrated embodiment of an optical interface clamp and a baseplate between which a portion of a subject is to be clamped for observation; and

FIG. 4 shows an illustrative embodiment of an assembled tail cuff device and a baseplate clamping a mouse tail in place for examination.

### DETAILED DESCRIPTION OF THE INVENTION

Certain terminology is used herein for convenience only and is not to be taken as a limitation on the present invention. Relative language used herein is best understood with reference to the drawings, in which like numerals are used to identify like or similar items. Further, in the drawings, certain features may be shown in somewhat schematic form.

The present systems and methods may include (a) in vivo labeling of targets (e.g. CTCs, infectious agents, etc.) in the bloodstream of the human and/or animal subject by IV injection of a fluorescently-labeled ligand (conjugate) which possesses specific binding affinity for a biomarker on the target, or alternately, IV injection of a previously-labeled target; (b) affixing the fiber optic sensor tip on, or closely adjacent to the skin surface of the human and/or animal subject in a position directly above, and substantially perpendicular to, a subcutaneous blood vessel, using an optical interface clamp device; (c) illumination of the target by the generation and transmission of coherent light at a wavelength optimized for the excitation of the fluorescent marker associated with the target; (d) detection of the fluorescent emission by the target; and (e) fluorescent signal amplification, processing and enumeration of fluorescent targets detected over time.

The system of the present technology includes the following components:

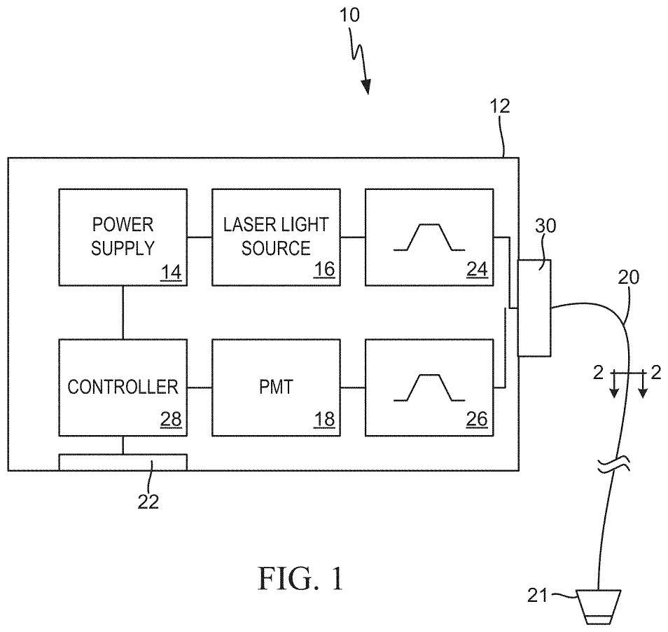

IVDx Device: The IVDx device 10, an example of which is shown schematically in FIG. 1, serves as an illumination device such as a coherent light source for excitation of the fluorescent dye associated with the target, as well the detector, signal amplifier and processor of the emitted fluorescent signal emanating from the stimulated target. Optionally enclosed in a single aluminum housing 12 (FIG. 1), the IVDx device 10 includes a power source 14 that selectively supplies electric power to energize a laser light source 16 which emits the specific required wavelength of excitation for the fluorescent dye used. The laser light emitted by the laser light source 16 is introduced to, and transmitted by a fiber optic bundle 20 to a cable sensor tip 21 from which the laser light is emitted to illuminate the interior of the blood vessel containing the fluorescent targets.

As described in detail below, the fiber optic bundle 20 includes a plurality of fiber sets with internal reflection to be optically isolated from each other. A first set of optic fibers transmits outbound laser light from the laser light source 16, and a second set transmits the fluorescence emitted by the fluorescing targets, as sensed by the cable sensor tip 21. Thus, the fiber optic bundle 20 is optically connected to both the laser light source 16 and a photomultiplier 18 for amplification of the incoming signal indicative of the fluorescence emitted by the fluorescing targets back to the IVDx. The IVDx can also include an interface 22 (e.g., RS-232 serial port, RJ-45 Ethernet port, short-range wireless communication port, etc.) for communicating with a laptop or other computer for data acquisition, data processing and instrument control. According to alternate embodiments, the necessary computer components can be disposed within the housing 12 and programmed to conduct the data acquisition, data processing and instrument control internally. The system additionally can be fitted with a narrow bandpass laser line filter 24 to ensure a narrow excitation spectrum from the laser light source 16. A photomultiplier tube (PMT) 18 is used for detection of the emitted fluorescence. The PMT 18 is fitted with an optical bandpass filter 26 which is selected based on the fluorescence spectra of the fluorescing targets such that collection of fluorescence is maximized while also maximizing the rejection of scattered light from the laser excitation. The PMT 18 used in this system is a counting PMT in which the analog signal from the photocathode is converted into a digital signal within the PMT 18. The digitized signal provides the input into a zero-dead-time counting system, interchangeably referred to herein as a controller 28, to maximize counting efficiency. The controller 28 can be operatively connected to communicate with an external computer for data processing, for example, via the interface 22.

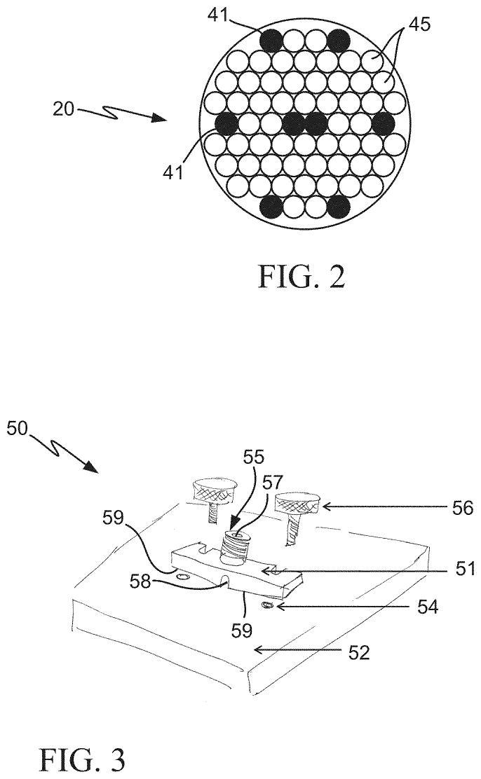

Fiber Optic Cable Bundle: The bifurcated fiber optic cable bundle 20 includes at least two sets of fibers, shown in the sectional view of FIG. 2. One set of fibers 41 (represented as shaded fibers) transmits the excitation light emitted by the laser to the cable sensor tip 21 for illumination of the interior of the blood vessel containing the fluorescent targets. The second set of fibers 45 transmits the fluorescence emitted by the fluorescing targets back to the IVDx device 10 for data acquisition and signal processing. Embodiments of the fiber optic cable bundle 20 comprise approximately thirty 100 μm fibers arranged such that 6-10 (8 excitation fibers are shown in FIG. 2) fibers are used for excitation and the remaining fibers are used for collection of fluorescence. The cable bundle 20 attaches to the housing 12 of the IVDx device 10 via an SMA 905 fiber connector 30 (FIG. 1) on the front panel of the device.

IVDx System Optical Interface Clamp Device: The IVDx system optical interface clamp 50 shown in FIG. 3 device serves to position and secure the sensor tip 21 of the fiber optic cable bundle 20 directly over, and within close proximity (optionally in contact with) the living tissue concealing the top of the blood vessel to allow optical stimulation of fluorescent targets circulating in the subject's blood, and detection of the emitted fluorescent signal resulting from stimulation of those fluorescent targets. As an example of such a device, a prototype tail cuff 51 was fabricated for use with laboratory mice, but a cuff configured to receive and closely approximate an appendage of any living creature to be examined is within the scope of the present disclosure. The mouse tail cuff is designed to precisely position the sensor tip 21 in a perpendicular orientation over one of the tail veins or arteries of the mouse. Although the present example relates generally to use with mice, rats, or other small animals having prominent surface blood vessels located in their tails, this device can be modified for use in larger animals and blood vessels located in the ears, limbs, or other structures. Similarly, this device can be reconfigured for use with human patients.

The mouse tail cuff device 51 is constructed from polyoxymethylene, commonly referred to as the trade name Delrin®, to provide durability and resistance to disinfecting agents. An arched notch 58 is defined by the tail cuff device 51 between planar surfaces 59 to receive the generally-tubular shaped mouse tail and firmly secure the mouse tail in place against a planar baseplate 52 while the planar surfaces 59 of the tail cuff device 51 rest against the planar surface of the baseplate 52. The planar baseplate 52 in the present example measures approximately 6 in.×8 in., and approximately 1 in. thick, and defines a plurality of internally-threaded holes 54 that releasably receive knurled knob fasteners 56, for example, which can be hand tightened and removed without the assistance of a tool for example to releasably couple the tail cuff device 51 to the baseplate 52 with the mouse tail received in the notch 58. An externally-threaded base 55 extends upward from an apex of the notch 58, defining an interior passage 57 that extends between the sensor tip 21, when the sensor tip 21 is coupled to the base 55, and the interior of the notch 58. The interior passage 57 can optionally include a light guide, lens or other suitable light transmission body.

The tail cuff clamp is configured as a flat rectangular clamp having a round central channel, referred to above as the notch 58, of a radius of 2.0 mm, for example, that runs across the bottom face, perpendicular to the long dimension of the clamp. The channel allows the clamp to be securely positioned over the top of the mouse's tail. Centrally located on the top face of the clamp is the perpendicular base 55 with threading to accommodates an SMA-905 fiber connector as the sensor tip 21, for example. This serves as the port through which the laser light passes as it is focused on the blood vessel, and simultaneously allows passage of light emitted from the stimulated fluorescent targets to the sensor tip for transmission back to the IVDx device 10. The threaded mount is configured such that when the fiber optic cable bundle is secured, the sensor tip 21 (FIG. 1), or the optional intermediary light guide, will be positioned within approximately 1 mm above the top of the channel. The clamp also has two slotted mounting holes for securement to the base plate via a pair of threaded screws.

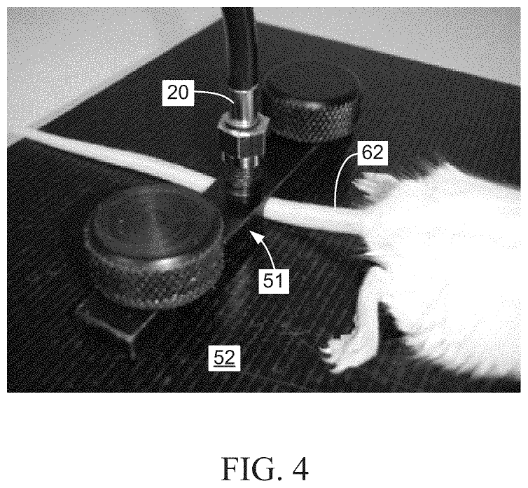

The mouse's tail is positioned within the central channel and secured by clamping it between the tail cuff clamp 51 on top, and the base plate 52 below, by use of two knurled knobs with threaded screws, placed through the slotted mounting holes in the tail cuff and screwed into the base plate. Prior to tightening the knurled knobs, the tail is positioned in a manner as to place the optical port directly over the target blood vessel. The assembled mouse tail cuff device 51 (with attached fiber bundle 20) and baseplate 52 securing a mouse tail 62 there between is shown in FIG. 4.

IVDx Data Processing Software: The IVDx software, optionally programmed into, or otherwise stored in a non-transitory, and optionally non-volatile computer-readable medium provided to the controller 28 (FIG. 1) controls the system, monitors the status of several system parameters and retrieves counting data from the photon counting system. Controls are provided for switching the PMT high voltage on and off, controlling laser power, activating and monitoring the PMT thermoelectric cooler and setting parameters for data acquisition [e.g. duration of acquisition and dwell time (accumulation time per data point)].

Data is automatically saved in the computer-readable medium and may be retrieved and displayed by this application at any time. Data can optionally be saved in a suitable text format to facilitate easy import of this data into other applications. A separate application can perform data analysis including peak identification and counting, but each of these applications can be easily integrated into a single application.

### EXPERIMENTAL/EXAMPLES

The following examples describe practical applications of the present invention in humans, laboratory animals and other research models. However, practical applications of the present invention are not limited to the examples described herein. It should be noted that various changes and modifications to the embodiments described herein will be apparent to those skilled in the art. Such changes and modifications may be made without departing from the spirit and scope of the present invention and without diminishing its attendant advantages. For example, various embodiments of the systems and methods may be provided based on various combinations of the features and functions from the subject matter provided herein.

### Example 1

### In Vivo Detection and Quantitation of Circulating Tumor Cells (CTCs)

In neoplastic disease, malignant cells often express unique biomarkers not found on normal (non-malignant) cells, or that are present at levels that are significantly different than those found on normal cells. The present invention exploits this principle by providing a means by which to detect and quantitate cancerous cells circulating in the bloodstream, so called circulating tumor cells (CTCs).

For human clinical applications, a diagnostic reagent including a fluorescent dye conjugated to an antibody or other ligand which specifically binds to a unique cancer biomarker is intravenously (IV) injected into a cancer patient. The fluorescent conjugate could also be comprised of fluorescent microspheres, including magnetic or non-magnetic material derivatized with an antibody or other ligand. Once in circulation, the fluorescent conjugate would then bind to, and label, the CTCs in the patient's bloodstream in vivo. In this manner, the present invention can be used to detect, quantitate, and monitor the number of CTCs in the bloodstream of the patient. Data can be collected at separate time points over an extended period, allowing the real-time monitoring of the numbers of CTCs in the bloodstream.

Alternately, in a research model, tumor cells expressing a unique cancer biomarker can be injected IV into a laboratory mouse or other animal to establish a tumor graft. A reagent comprising a fluorescent dye conjugated to an antibody or other ligand which specifically binds to the unique cancer biomarker is then injected IV into the animal. The fluorescent conjugate could also be comprised of fluorescent microspheres, including magnetic or non-magnetic material derivatized with an antibody or other ligand. The fluorescent conjugate would then bind to, and label, the CTCs in the bloodstream of the animal in vivo. The present invention can be used to detect, quantitate, and monitor the number of CTCs in the bloodstream of the animal. Data can be collected at separate time points over an extended period, allowing the real-time monitoring of the numbers of CTCs in the bloodstream. In this example, use of the system and methods of the invention provides a convenient, in vivo, minimally invasive model for the progression of cancer in a laboratory animal.

### Example 2

### In Vivo Detection and Quantitation of Metastatic CTCs

In a research application, tumor cells expressing a unique a cancer biomarker can be injected into a tissue site in the subject other than the blood to establish a focal tumor graft. The ability of the cells of the resulting tumor to extravasate into the bloodstream as CTCs can then be determined, over time, by detection and quantitation of the resulting CTCs using the system and methods of the present invention as described in example 1 above. In this example, use of the system and methods of the invention provides a convenient, in vivo, minimally invasive model for metastatic cancer in a laboratory animal.

### Example 3

### In Vitro Labeling of CTCs

As an alternative to the in vivo CTC labeling method described in example 1 above, it may be advantageous to conduct the labeling procedure in vitro, in a manner external to the research animal, prior to injection of the CTCs. In such a case, the tumor cells are first be incubated with the fluorescent conjugate in vitro, followed by removal of excess or unbound conjugate, and the resulting labeled tumor cells are injected into the research animal via the IV or other routes. The present invention is then used to detect, quantitate, and monitor the resulting labeled CTCs in the bloodstream of the animal. Applications of this method include, but are not limited to, studies of the clearance of CTCs from the bloodstream, extravasation of CTCs into the bloodstream from injection sites external to the bloodstream, and anti-cancer agent efficacy studies.

### Example 4

### Study of Tumorigenic Processes

Metastasis is the primary cause of mortality in cancer patients. The epithelial mesenchyinal transition (EMT) in tumor cells is thought to play a critical role in the development of tumorigenic cancer stem cells (CSCs), which have been implicated in the development of metastatic tumors. There is evidence that CSCs possess a unique biomarker (CSC biomarker). Utilizing the in vivo animal cancer models described in examples 1, 2 and 3 above, the present invention can be used to identify and monitor CSCs by utilizing a fluorescent conjugate which specifically targets the CSC biomarker. This application of the invention allows for detection of EMT events and study of the processes leading for tumorigenesis.

### Example 5

### Evaluation of the In Vivo Efficacy of Anti-Cancer Agents

Utilizing the in vivo CTC detection and quantitation methods, and/or animal cancer models described in examples 1, 3 and 4 above, the present invention can be used to monitor the efficacy of anti-cancer agents by determining the effect of treatment on the change (delta) in the number of CTCs in the bloodstream of the subject at various post-treatment time points. A negative delta, or decrease in the number of CTCs would be indicative of anti-cancer efficacy, while a positive delta, or increase in CTCs would indicate a lack of effectiveness of the agent. In this application, the present invention provides clinicians and researchers with a system and methods for real-time monitoring of the effects of anti-cancer treatment on the progression of the disease. In a similar manner, this application can be extended to use for the identification of anti-tumor agents effective against CSCs.

### Example 6

### Detection and Quantitation of Intrinsically Fluorescent Cells

For the study of normal or disease physiologic processes, it may be advantageous to inject into the bloodstream of the research animal cells which are intrinsically fluorescent. Such cells may be created by genetic engineering by insertion of genetic material into the cells which encodes for the production of green fluorescent protein (GFP), or by other means. In an application, the present invention could be used to detect, quantitate and monitor such intrinsically fluorescent cells in the bloodstream of a research animal. Applications of this method include, but are not limited to, oncology, anti-cancer efficacy, and immunological studies.

### Example 7

### Detection and Quantitation of Blood Borne Infectious Agents

In an application, the present invention could be used to monitor a blood-borne infection in a human clinical patient, or in an animal model. In this application, the fluorescent conjugate would contain an antibody or other ligand which would specifically bind to a unique biomarker on the infectious agent in the bloodstream of the subject. Applicable infectious agents include, but are not limited to, bacteria, viruses, fungal cells, and protozoa. The system could then be used to monitor the pathogenesis and progression of the infection, and would thusly provide a convenient, in vivo, minimally invasive method for the diagnosis, monitoring and study of infection processes and progression.

### Example 8

### Evaluation of the In Vivo Efficacy of an Anti-Infective Agent

Utilizing the in vivo clinical monitoring system or animal infection model described in example 7 above, the present invention could be used to monitor the efficacy of anti-infective agents by determining the effect of treatment on the change (delta) in the number of infectious particles circulating in the bloodstream of the subject at various time points post-treatment. A negative delta would be indicative of efficacy of the anti-infective agent, while a positive delta would indicate progression of the infection and lack of effectiveness of the agent. In this way, the present invention would provide clinicians and researchers with a system and methods for real-time monitoring of the effects of anti-infective treatment on the progression of the infectious disease.

### Example 9

### Evaluation of the Pharmacokinetics of Particle-Based Pharmaceutical Agents or Drug Delivery Systems

In an application, the present technology could be used to monitor pharmacokinetic (PK) parameters of particle-based pharmaceutical agents or drug delivery systems including, but not limited to, liposomal formulations, dendrimers, microencapsulated formulations and high molecular weight polymeric drug carriers. In this application, a fluorescent dye is conjugated to, or incorporated into, the pharmaceutical agent or drug delivery vehicle, allowing the agent to be detected and quantitated in the bloodstream using the system and methods of the present invention. Real-time monitoring of circulating concentrations of the agent would provide data useful for the calculation of various PK metrics such as, but not limited to, residence time and clearance, maximum circulating concentration (Cmax), and elimination half-life (T½). A related application of the present invention extends to toxicological studies.

### Example 10

### Characterization of Normal Blood Cells

In an application, the present invention could be used to monitor normal cells in the bloodstream of the subject. Applicable cell types include, but are not limited to cells having a targetable surface receptor such as erythrocytes, and leukocytes including lymphocytes, monocytes, neutrophils, basophils, and eosinophils.

### Example 11

### Characterization of Immune System Disorders

In an application, the present invention could be used to characterize and monitor disorders of the immune system in human clinical patients and laboratory animal models in which abnormal cell types having a unique targetable surface receptor are involved, or in cases in which atypical numbers of normal cells are present in the bloodstream.

### Example 12

### Other Applications of the Invention

In another application, the present invention could be used for the detection, quantitation and monitoring of any fluorescent, particulate, material in the bloodstream of human patients or laboratory animals.

Illustrative embodiments have been described, hereinabove. It will be apparent to those skilled in the art that the above devices and methods may incorporate changes and modifications without departing from the general scope of this invention. It is intended to include all such modifications and alterations within the scope of the present invention. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A device for excitation of a fluorescent component associated with a predetermined target circulating in blood flowing through a blood vessel of a living being, the device comprising:
a clamping device for securing an appendage with the blood vessel through which the blood is flowing against a baseplate;
a coherent light source that emits light that causes excitation of the fluorescent component forming a conjugate with the predetermined target in the blood stream;
a fluorescence sensor that senses a fluorescent effect exhibited by the fluorescent component forming a portion of the conjugate in response to excitation of the fluorescent component by the light emitted by the coherent light source;
an emitted light transmission device that transmits the light emitted by the coherent light source between the coherent light source and the clamping device to impart the light on the appendage containing the blood vessel secured by the clamping device; and
a fluorescence transmission device that transmits the fluorescent effect exhibited by the fluorescent component of the conjugate between the clamping device and the sensor.

2. The device of claim 1, wherein the emitted light transmission device comprises a plurality of transmitted-light optical fibers and the fluorescence transmission device comprises a plurality of fluorescence optical fibers, and the transmitted-light optical fibers and the fluorescence optical fibers are optically isolated from each other, and bundled together as part of a common fiber bundle.

3. The device of claim 2, wherein the common fiber bundle comprises a cable sensor tip that is to be releasably coupled to a base provided to the clamping device.
